(19) United States
(12) Patent Application Publication
(10) Pub. No.: US 2008/0188902 A1
(43) Pub. Date: Aug. 7, 2008

(54) CONTACT MODULE FOR A MEDICAL IMPLANT

(76) Inventor: Marcel Starke, Berlin (DE)

Correspondence Address: DALINA LAW GROUP, P.C., 7910 IVANHOE AVE. #325, LA JOLLA, CA, 92037, US

(21) Appl. No.: 12/022,837

(22) Filed: Jan. 30, 2008

(30) Foreign Application Priority Data
Feb. 7, 2007 (DE) 10 2007 006 088.4

(51) Int. Cl. A61N 1/375

(52) U.S. Cl. 607/36

(57) ABSTRACT

The present invention relates to an electrical connection between the individual components of active medical implants, in particular between a voltage source and an electrical consumer. The voltage source is electrically connected to the electrical consumer via a spring contact block. The electrical connection between the voltage source and the electrical consumer is produced by the spring contact block, in that the contacting face of the spring contact rests on a counter contact face of the consumer or the voltage source and a pressure force generated by the spring restoring force of the spring contact is exerted on the counter contact face, the counter contact face being implemented as essentially level and situated parallel to the surface, to which it is fastened.

This application takes priority from German Patent Application DE 10 2007 006 088.4, filed 7 Feb. 2007, the specification of which is hereby incorporated herein by reference.

### BACKGROUND OF THE INVENTION

1. Field of the Invention

The present invention relates to an electrical connection between the individual components of active medical implants, in particular between a voltage source and an electrical consumer.

2. Description of the Related Art

Active medical implants, above all medical pulse generators, cardiac pacemakers, or cardioverter-defibrillators, are systems which must have an extremely low breakdown probability and/or error rate, because they fulfill an important, sometimes vital task for the health of a patient. For this reason, particularly wide-ranging quality assurance measures are performed in the production of medical pulse generators.

In addition to the continuous performance of quality checks during the production process, particularly high-quality electronic components and materials are also used in pulse generators.

Active medical implants, to which the present patent application relates, typically have a housing in which electrical components of the implant are housed. Main components are a voltage source, i.e., for example, a battery, as well as electrical consumers which are to be supplied with energy by the voltage source. The electrical consumers are typically constructed in the form of circuits, which have, for example, a circuit board and electrical and electronic components mounted thereon, such as integrated circuits. The circuits form pulse generators for generating electrical stimulation pulses, for example, which are to be delivered to a chamber of a heart to stimulate a contraction of the particular chamber.

To ensure the error-free operation of pulse generators least for the planned operating life even after implantation, they are enclosed by a housing which is at least liquid-tight, but is typically also hermetically sealed, which prevents the penetration of blood and tissue fluid into the interior of the housing.

The housing comprises titanium, for example, and is typically assembled from two half shells, which are connected tightly to one another after the implant is manufactured, so that the housing is subsequently closed.

Once closed, the housing is only to be opened with force, and replacing individual components, such as the voltage source, after a specific time is not intended. Rather, implantable cardiac pacemakers, cardioverters, and defibrillators are intended for onetime use and are replaced by a new device after expiration of the permissible operating time. For this purpose, explanation of the old device and the implantation of a new device are necessary. Because a replacement of system components is not intended for such implants and is even legally forbidden due to safety-technology considerations, the individual components of the implants according to the prior art, in particular the components voltage source and electrical consumers, but also other components, are electrically connected to one another by fixed connections.

Typically, fixed electrical connections between individual components or lines are produced by soldering or welding, rarely by crimping or another type of friction-locked or formfitting connection.

Because intracardial heart therapy has developed in the meantime to a standard method which has proven itself millions of times worldwide, it is advisable for reasons of cost to automate the production method of the implants. The construction of current electromedical implants may be simplified as described in the following. Up to this point, all components, such as a battery, a circuit, possibly a telemetry unit or the like, have usually been housed adjacent to one another in the implant housing. The implant housing itself typically has a flat, elongate contour having rounded edges and is typically formed by two half shells having a type of snap mechanism made of interlocking edges.

Such a configuration, in which the individual components are usually electrically connected to one another by a fixed connection, in particular by soldering, has the disadvantage, however, that multiple manufacturing axes must be controlled during the mounting of the individual components. This makes automation difficult and results in higher costs.

The electrical connection between power-consuming components and the battery must additionally consider the following problem: during the discharge of the battery, there is a slight volume increase of the battery as a result of the basic electrochemical reaction. This swelling of the battery caused by discharge may result in problems in the electrical connection between the battery and the circuit or another component connected to the battery.

### BRIEF SUMMARY OF THE INVENTION

The object of the present invention is to optimize the construction of the implant from the aspect of a production method which is as simple and automatable as possible, without the reliability of the implant thus being impaired.

This object is achieved according to the present invention by an active medical implant which has a housing, which encloses at least two electrical components, e.g. a battery and a circuit, liquid-tight. The implant is distinguished in that
at least two electrical components are electrically connected to one another via a spring contact block,
at least one of the electrically conductive components having at least one counter contact face, which is implemented as a flat, electrically conductive surface element of a surface of the component,
the spring contact block having at least one electrically insulating contact carrier and at least one conductive, elastic spring contact retained thereby,
which has at least two electrically conductive contacting faces, which are electrically connected to one another, and
which is implemented in such a way that at least one of the contacting faces is deflected elastically sprung under pressure on the contacting face and builds up a defined spring restoring force,
an electrical connection being produced between two electrically conductive components by the spring contact block in such a way that the contacting face of the spring contact rests on at least one counter contact face of at least one component and exerts a pressure force generated by the spring restoring force of the spring contact on the counter contact face.

As described above, there is a slight volume increase of a battery during the discharge of the battery. This may result in problems in typical pulse generators. An advantage of the present invention is that swelling of the battery may be compensated for especially well with the aid of the spring contacts of the spring contact block, because relative movement is possible between the circuit and the battery.

Therefore, the contact carrier may preferably form a mechanical stop, which prevents relative movements perpendicular or horizontal to the upper surface of the contact carrier. Mechanical strains which may arise either through the volume increase of the battery or external strain in use and during production may be dissipated on the relatively stable power source, which is also preferably a battery, which prevents damage to the consumer and its components.

A further advantage of the present invention comprises simplifying the manufacturing process of medical pulse generators by avoiding typical, fixed electrical connections, in particular soldered connections, between individual components.

Instead, an electrical connection occurs automatically between the electrically conductive components of the implant according to the present invention upon joining of the particular components due to the spring restoring force of the contact springs between the contacting faces of the spring contacts and the counter contact faces on the corresponding components.

According to an especially preferred embodiment variant of the present invention, the spring contact block has multiple spring contacts, which have an essentially flat cross-section.

The spring contacts preferably have a curvature which exclusively runs in a plane which runs perpendicularly to a surface of the spring contact, preferably the flat side. The curvature preferably has a radius of less than 2 mm.

According to an especially preferred embodiment, the contacting faces of the spring contacts are curved convexly in the direction of the counter contact faces.

The spring contacts are preferably produced from copper-containing alloys and have a contacting face which comprises an alloy containing gold or nickel. The spring contacts preferably have a flat cross-section of less than 1 mm and are held by the contact carrier in a curved state.

The electrical component which has the counter contact faces preferably has a number of counter contact faces corresponding to the spring contacts of the spring contact block. The counter contact faces are preferably flat elements which are either embedded directly in a surface of the corresponding electrical component, or they are held by a counter contact carrier which is in turn embedded in a surface of the corresponding component.

According to an especially preferred embodiment variant, the contacting faces of the spring contacts and/or the counter contact faces are coated with an alloy containing gold or nickel to prevent an increase of the electrical resistance as a result of corrosion.

According to an especially preferred embodiment variant, the electrical consumer and the voltage source are electrically connected to one another via the spring contact block.

The spring contact block is especially preferably a surface-mounted component and is soldered directly to the circuit board and thus electrically connected thereto. According to this especially preferred embodiment variant, the battery has at least one counter contact face.

The medical implant is preferably constructed in such a way that the housing of the implant is formed by two half shells which are joined and welded at the end of the production process. The components of the implant located in the housing are thus enclosed liquid-tight.

The voltage source is preferably housed in one of the two half shells, and the electrical consumer in the particular other half shell. Both the voltage source and also the electrical consumer are preferably connected fixed to the particular half shell.

According to a preferred design of the present invention, the implant housing comprises two half shells, one of the half shells simultaneously being a component of the battery housing.

If, according to the especially preferred embodiment variant, the voltage source and the electrical consumer are electrically connected to one another via the spring contact block, the spring contact block and the counter contact faces are situated inside the particular half shell in such a way that the spring contacts are pre-tensioned by the counter contact faces through the joining of the two half shells. The spring restoring force thus resulting causes the contacting faces of the spring contacts to be pressed on the counter contact faces, and an electrical connection to be produced between the voltage source and the electrical consumer.

In addition to the especially preferred configuration of the spring contact block and the counter contact faces suggested up to this point, numerous further configurations of spring contact block and counter contact faces correspond to different embodiment variants of the present invention.

According to one of these embodiment variants, both the electrical consumer and also the voltage source have counter contact faces which are situated in such a way that they press in the spring contacts of the spring contact block upon the assembly of the two half shells. Both the counter contact faces of the electrical consumer and also the counter contact faces of the battery are thus electrically connected to the spring contact block via the contacting faces of the spring contacts. An electrical connection between the voltage source and the electrical consumer is thus produced.

If an electrical connection occurs according to this embodiment variant, the spring contact block is not connected fixed to any of the components. To prevent a horizontal displacement of the spring contact block, which may have the result that the contacting faces of the spring contacts no longer rest on the counter contact faces, according to a preferred embodiment variant, at least one of the two half shells or one of the two electrical components has a retainer which prevents a corresponding movement of the spring contact block.

According to a further embodiment variant, the spring contact block is connected, as a surface-mounted component, fixed to the battery and electrically to the poles of the battery. In this case, the electrical consumer preferably has counter contact faces. The counter contact faces are situated in such a way that they press in the spring contacts of the spring contact block upon the assembly of the two half shells, by which an electrical connection is produced between the battery and the electrical consumer.

According to further embodiment variants, one or more spring contact blocks also produce electrical connections between multiple electrical components of the electrical consumer. In this way, medical implants may be assembled especially easily like a sandwich without soldering, welding, or the like.

A further aspect of the present invention relates to a method for producing the medical implant described above. In this context, several particularly advantageous method steps for producing the above-mentioned object of the present invention are cited in the following.

Accordingly, the voltage source, which is connected fixed to one of the half shells by gluing, forms the base for the mounting. The electrical consumer having at least one circuit on a circuit board is located in the other half shell, preferably connected fixed thereto. The spring contact block is preferably already soldered on the circuit board as a surface-mounted component.

Both half shells are joined in one of the following method steps. The spring contacts of the spring contact block are pressed in and pre-tensioned. The spring restoring force resulting therefrom presses the contacting faces of the spring contacts onto the counter contact faces of the voltage source, by which an electrical connection is produced.

These method steps are used especially advantageously for flanged housings. After the joining of the half shells, in which the electrical connection has been produced automatically between the electrical voltage source and the circuit, the two half shells are preferably sealed airtight and liquid-tight.

### BRIEF DESCRIPTION OF THE DRAWINGS

In the following, several especially preferred embodiment variants of the present invention are explained in connection with FIGS. 1 through 9.

FIG. 1 is a top view of the two housing halves of the implant in an open state.

FIG. 2 is a perspective illustration of the electrically conductive counter contact faces.

FIG. 3 is a perspective illustration of the spring contact block.

FIG. 4 is a cross-sectional illustration of the electrical connection between voltage source and electrical consumer.

FIG. 5 is a cross-sectional illustration of the medical implant according to the main embodiment variant, having a spring contact block fastened to the circuit board as a surface-mounted element.

FIG. 6 is a cross-sectional illustration of a second embodiment variant of the present invention.

FIG. 7 is a cross-sectional illustration according to a third embodiment variant of the present invention.

FIG. 8 is a cross-sectional illustration according to a fourth embodiment variant of the present invention.

FIG. 9 is a cross-sectional illustration according to a fifth embodiment variant of the present invention.

### DETAILED DESCRIPTION OF THE INVENTION

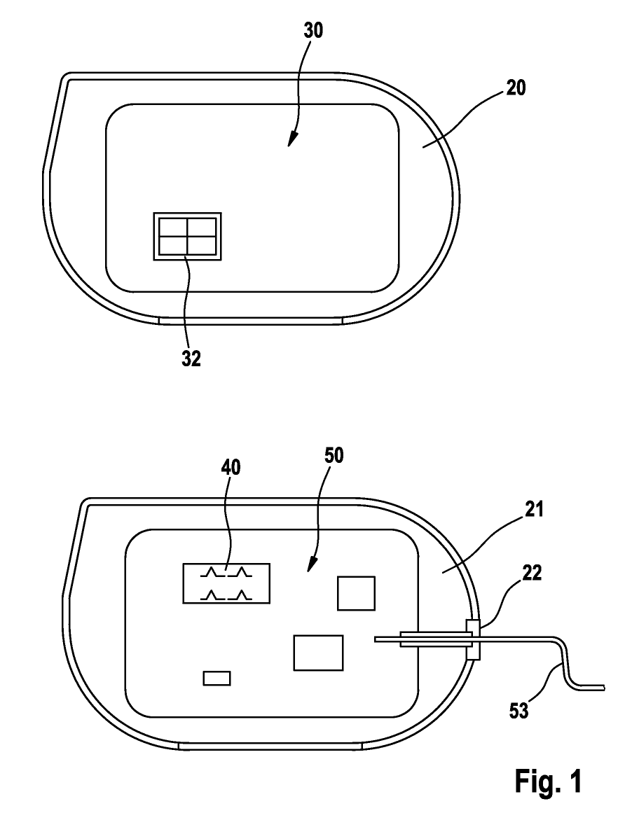

FIG. 1 shows the basic components of an active medical implant. The implant comprises two housing halves which are implemented as half shells 20 and 21. A battery 30 is housed as the voltage source in the first half shell 20 and preferably connected fixed to the half shell 20 by gluing, for example.

The second half shell comprises an electrical consumer 50.

To produce an electrical connection between battery 30 and consumer 50 upon joining of the two half shells in the scope of a production method for the implant, the battery comprises four counter contact faces according to this embodiment variant, which are implemented as flat, electrically conductive surface elements and which are electrically connected to the battery 30.

The electrical consumer 50 has a spring contact block 40, which is in turn electrically connected to the electrical consumer 50.

When the two half shells are joined in a later production step, the electrical connection is thus also produced between the electrical consumer and the battery, as described in greater detail in the following figures. The consumer is thus made capable of processing electrical signals and exchanging or transmitting them with or to a location to be treated or monitored via a conductor strand 53, which is led through a feed through 22 of the half shell 21.

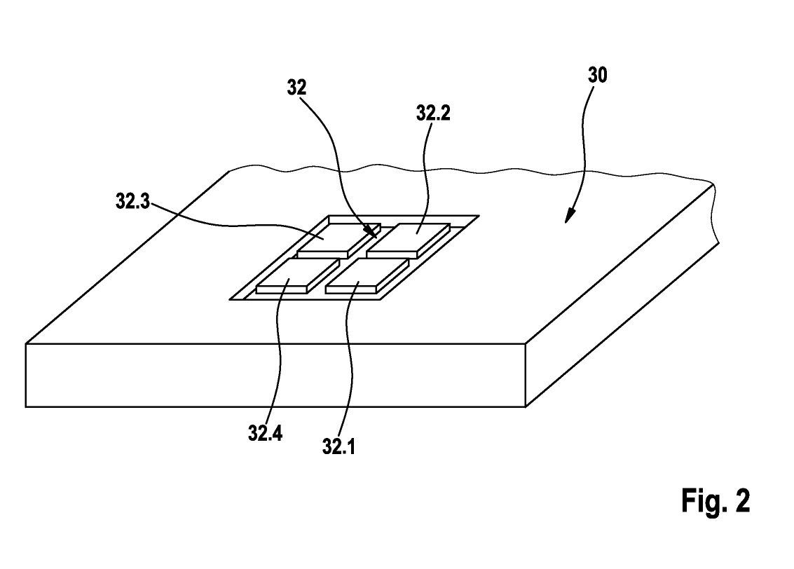

FIG. 2 shows a detail of the battery 30 having a counter contact carrier 32. According to this embodiment, four counter contact faces 32.1, 32.2, 32.3, and 32.4 are embedded in the counter contact carrier 32. These counter contact faces 32.1, 32.2, 32.3, and 32.4 are electrically connected to the poles of the battery 30. Each of the counter contact faces 32.1, 32.2, 32.3, and 32.4 forms a flat, electrically conductive surface element of the battery.

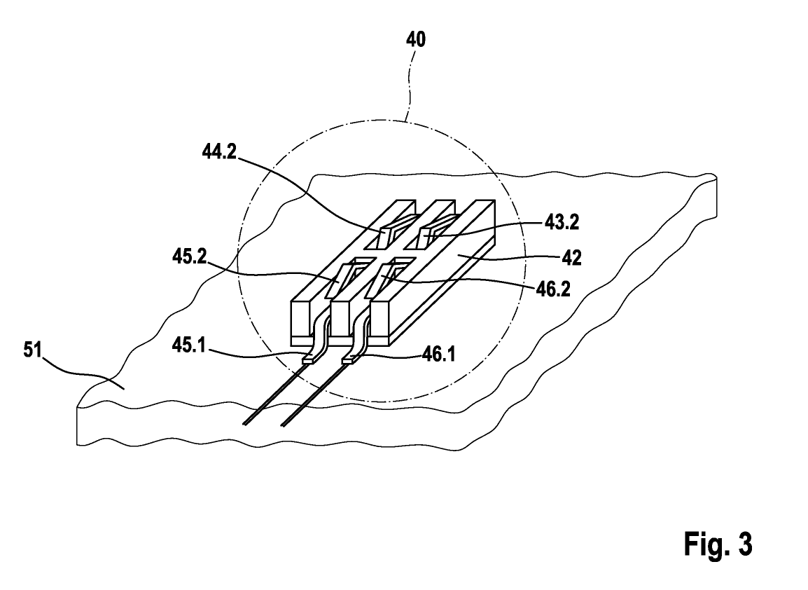

FIG. 3 shows a perspective partial view of the consumer 50, to which the spring contact block 40 is attached. The spring contact block 40 is preferably a surface-mounted component which is soldered directly onto a circuit board 51 of the electrical consumer 50. The spring contact block 40 comprises an electrically insulating contact carrier 42, in which four spring contacts 43, 44, 45, and 46 are retained according to this embodiment variant. Each of the spring contacts has at least two conductive contacting faces 43.1, 43.2, 44.1, 44.2, 45.1, 45.2, 46.1, and 46.2. One contacting face 43.1, 44.1, 45.1, and 46.1 of each spring contact is electrically connected to the circuit board 51 according to this embodiment variant. The second contacting face 43.2, 44.2, 45.2, and 46.2 of each spring contact, which is electrically connected to the first contacting face, projects upward out of the contact carrier 42.

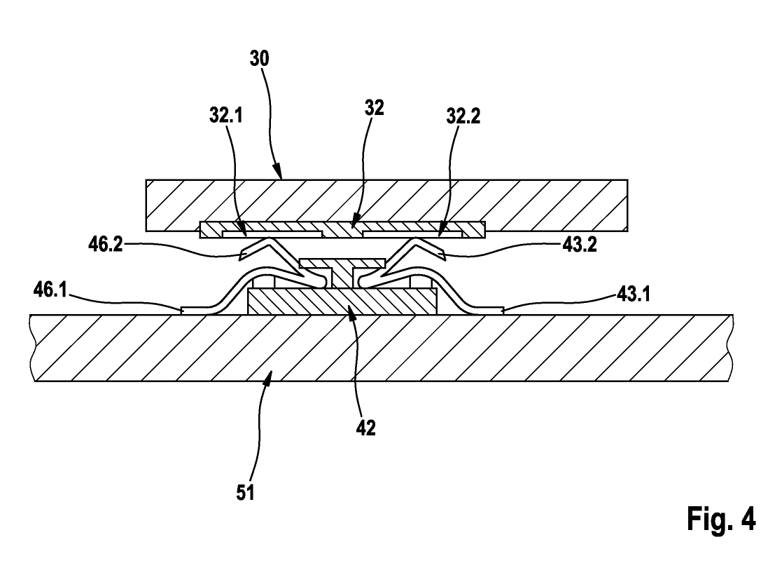

FIG. 4 is a cross-sectional illustration of the electrical connection between voltage source and electrical consumer. From bottom to top, the illustration shows the contact carrier 42, which is attached to the circuit board 51. The contact carrier 42 holds the two spring contacts 45 and 46. These each have, as already described above, two contacting faces 45.1, 45.2 and 46.1, 46.2, which are each electrically connected to one another. The spring contacts are electrically connected to the circuit board 51 at the contacting faces 45.1 and 46.1 and are preferably soldered thereon. The two counter contact faces 32.1 and 32.2, which are implemented as flat, are located above the contacting faces 45.2 and 46.2, electrically connected to the contacting faces. These counter contact faces are electrically connected to the poles of the battery 30. The counter contact faces 32.1 and 32.2 each form two electrically conductive surface elements of the battery 30. Preferably—as shown in this figure—the two counter contact faces are embedded in a counter contact carrier 32, which is in turn embedded in the surface of the battery 30 and thus connected fixed thereto.

The electrical connection between the contacting faces and the counter contact faces occurs in that upon joining of the two half shells 20 and 21, the two counter contact faces 32.1 and 32.2 exert a force on the contacting faces 45.2 and 46.2. The spring contacts 45 and 46 are thus pre-tensioned and produce a permanent electrical connection between the contacting faces 45.2, 46.2 and the counter contact faces 32.1 and 32.2 with the aid of the spring restoring force thus generated. The supply of the circuit board 51 and thus also of the electrical consumer 50 as a whole by the battery 30 is thus ensured.

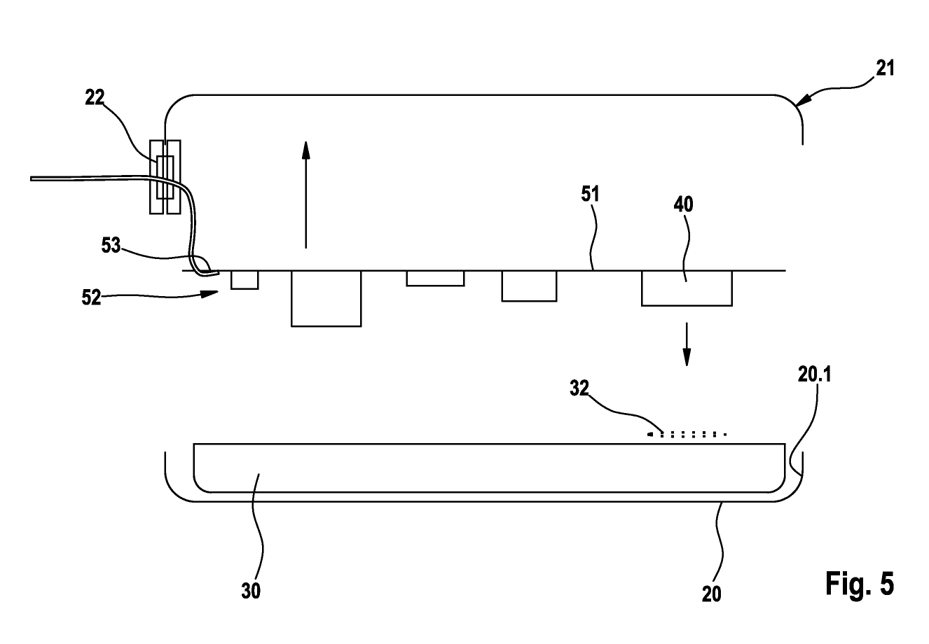

FIG. 5 shows the cross-sectional illustration of a preferred embodiment variant of the medical implant which has the half shells 20 and 21. The two half shells 20, 21 are implemented as snap shells having interlocking edges.

Half shell 20, which is used as the base in an especially preferred production method of the medical implant, comprises the battery 30 as the voltage source, which is materially bonded to the half shell 20 by gluing. A small distance 20.1 is preferably provided between the peripheral narrow side of the battery 30 and the half shell 20, which gives the battery room for an expansion occurring during the discharge procedure. The counter contact carrier 32, in which the counter contact faces are embedded, is fastened in the surface of the battery 30 facing inward in relation to the implant. The counter contact carrier 32 is electrically connected fixed to the poles of the battery 30 as a surface-mounted component.

In addition to the battery as the first electrical component of the implant, an integrated circuit mounted on a circuit board 51 is housed in the half shell 21 as the second electrical component of the implant. Further components of the active medical implant are possibly housed therein. The circuit board 51 is preferably connected fixed to the housing half 21. According to this embodiment variant, further surface-mountable components 52 are soldered on the circuit board 51, which contains the integrated circuit. The integrated circuit in the circuit board 51 and, if present, all further functional components which are electrically connected to the battery 30 are referred to jointly as the electrical consumer 50 in this patent application.

As recognizable in the cross-sectional illustration in FIG. 5, the spring contact block 50 is soldered onto the circuit board 51 as a surface-mounted component in such a way that the spring contact block 40 is located directly above the counter contact carrier 32. The first end of the conductor strand 53 is electrically connected to the circuit board 51 according to this embodiment variant. The conductor strand 53 is led through the feed through 22 of the housing 21, so that electrical signals may be conducted from the circuit board 51 to the second end of the conductor strand 53, which is located outside the housing 21.

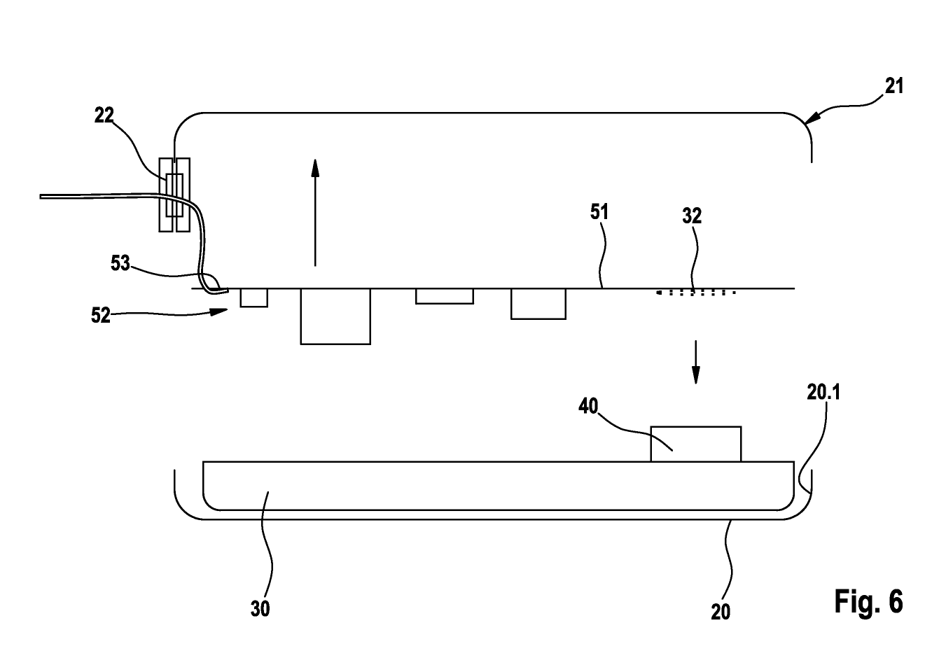

A further alternative configuration having a single-axis component structure is schematically shown in FIG. 6. In its main features, it corresponds to the configuration of circuit board 51 and battery 30 already presented in FIG. 5. In contrast to the embodiment from FIG. 5, the counter contact carrier 32 in which the counter contact faces are embedded is connected fixed to the circuit board 51, and the spring contact block is connected fixed to the battery 30 and electrically to the poles of the battery 30.

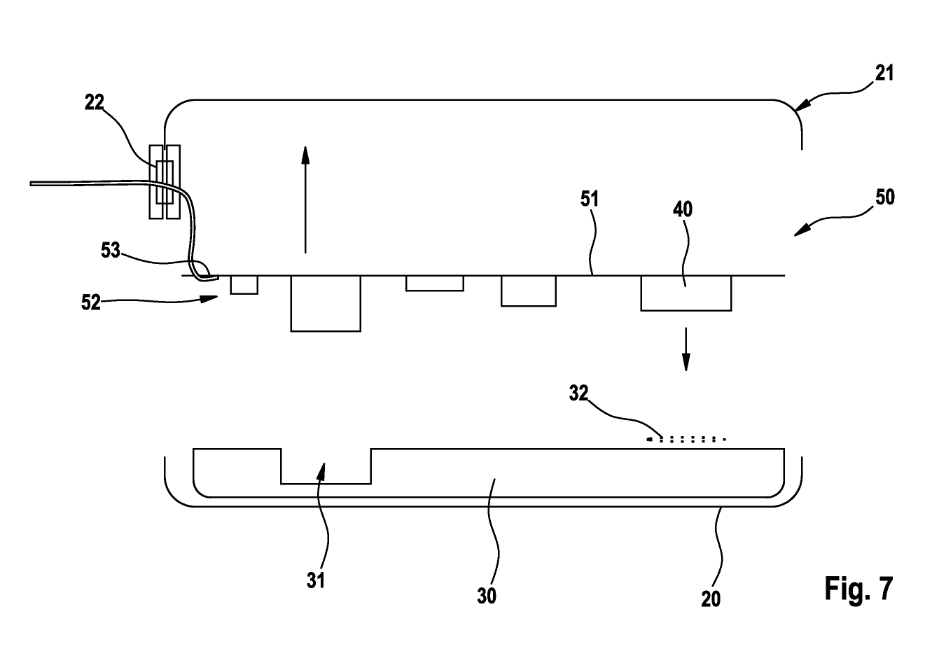

FIG. 7 shows a further design variant of the object of the present patent application. The configuration of circuit board 51 and battery 30 as well as of spring contact block 40 and counter contact carrier 32 corresponds to the embodiment variant from FIG. 5. In contrast to FIG. 5, the battery 30 has at least one recess 31 according to this design, which allows the electrical components 52, which are soldered on the circuit board 51 and have especially large dimensions in the vertical direction, to partially find room in the recess(es) 31 of the battery 30 and thus allow a more compact construction of the implant.

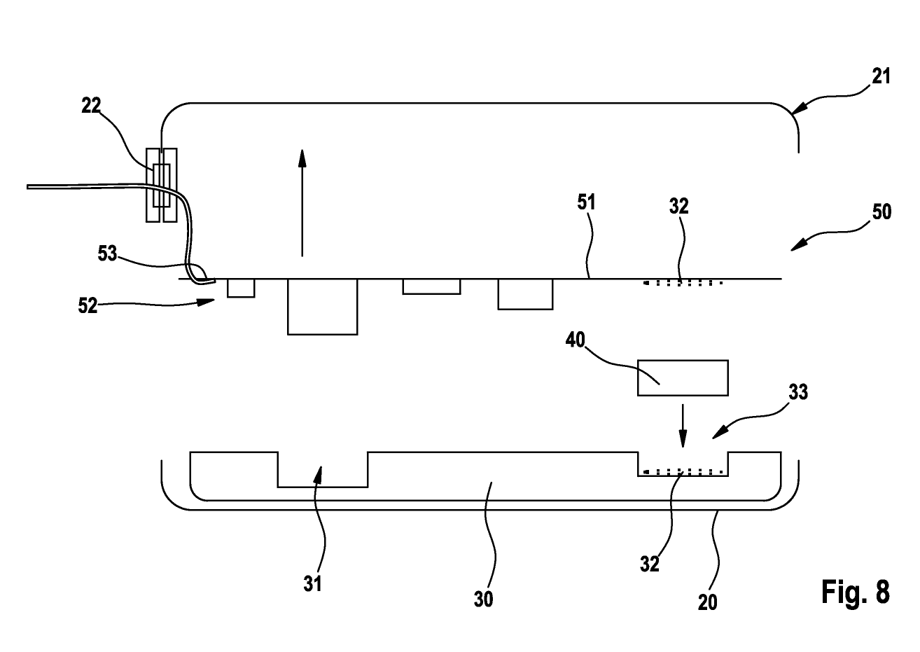

FIG. 8 shows a further design variant of an active implant. In contrast to the embodiment variants illustrated in FIGS. 5 through 7, the implant has two counter contact carrier 32 and 34 in this design. The counter contact carrier 34 is connected fixed to the circuit board. The counter contact carrier 32 is located on the floor of a recess 32 in the battery 30 and is connected fixed to the battery. The spring contact block 40 is not a surface-mounted component in this embodiment variant and/or is neither connected to the circuit board 51 nor to the battery 30 in this design. Rather, the spring contact block is inserted into the recess 33 of the battery 30 during the production method of the implant. If the counter contact faces of the circuit board, which are embedded in the counter contact carrier 34, are pressed onto the contacting faces of the spring contacts by the joining of the two half shells during a later production step, the spring contacts are pre-tensioned. The spring restoring force resulting therefrom thus causes an electrical connection between the first contacting faces of the spring contacts and the counter contact faces of the circuit board as well as the second contacting faces of the spring contacts and the counter contact faces of the battery, which are enclosed by counter contact carrier 32.

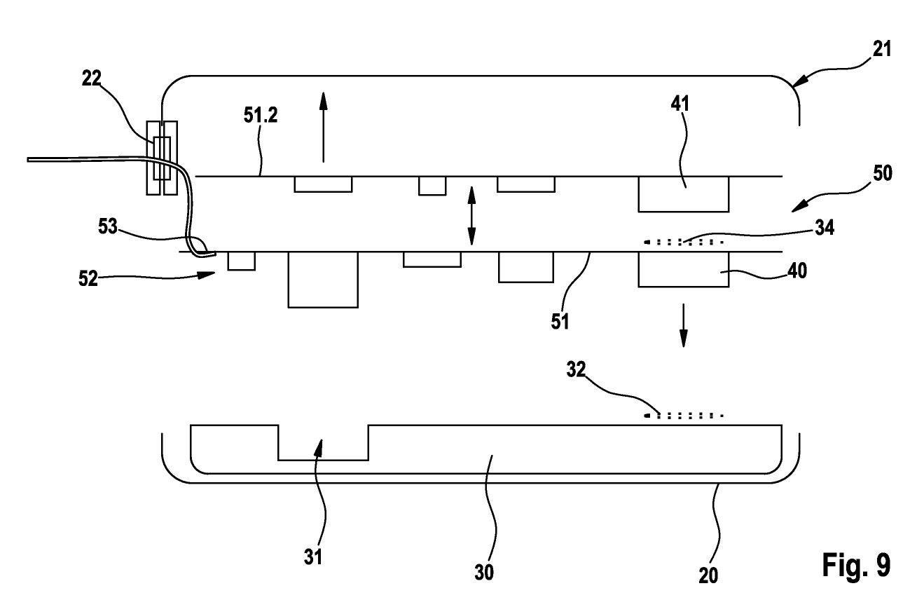

FIG. 9 shows a further embodiment of an active implant. Accordingly, according to the same principle of an electrical connection produced with the aid of spring contact block and counter contact faces, further modules of electrical consumers may also be connected directly or indirectly via the circuit board 51 to the poles of the battery 30. According to the design shown here, a counter contact carrier 34 is attached on the side of the circuit board 51 facing away from the battery and electrically connected to the circuit board 51. Circuit board 51.2 is located above the circuit board 51 as a further functional group of the medical implant. The spring contact block 41 is connected fixed and electrically to the circuit board 51.2 on the side of the circuit board 51.2 facing away from the circuit board 51. The joining of the half shells causes, both in the spring contacts of the spring contact block 40 and also in the spring contacts of the spring contact block 41, a spring restoring force, by which an electrical connection is produced both between battery and circuit board 51 and also between circuit board 51 and circuit board 51.2.

In all of the cited embodiment variants, the counter contact 40 has an insulating contact carrier 42, which is used on one hand for insulation between the spring contacts 43, 44, 45, and 46, and also, on the other hand, offers the spring contacts a guide perpendicular to the upper surface of the contact carrier 42 and positions the spring contacts. Furthermore, the main body may also, as a mechanical stop, prevent the relative movements between the components—i.e., for example, the voltage source 30 and the circuit board 51 and/or 51.2—perpendicular and/or horizontal to the surface of the contact carrier 42. These relative movements result, for example, from the swelling of the battery or also in the event of degrees of freedom in the internal construction. To prevent these relative movements, the contact carrier may have a defined height, for example, or may have one or more lateral stops on the edges of the upper surface, for example, which prevent the counter contact faces 32.1, 32.2, 32.3, and 32.4 from losing the contact to the spring contacts 43, 44, 45, and 46. However, the stop may also be designed in such a way that the counter contact carriers 32 and/or 34 lose the contact to the contact carriers 40 and 41. The integrated circuits or electrical components 52 are thus protected from mechanical strain and the service life of the medical implant is extended.

The medical implants illustrated in FIGS. 1 through 9 only represent especially preferred embodiments of the object of the present application. Further embodiments result from the scope of the patent claims.

What is claimed is:

1. A medical implant having a housing, which encloses at least two electrical components (30, 50, 51, 51.2) liquid-tight, comprising:
at least two electrical components electrically connected via a spring contact block (40, 41);
at least one component selected from the at least two electrical components having at least one counter contact face (32, 34) which is implemented as a flat, electrically conductive surface element of a surface of the at least one component;
the spring contact block (40, 41) having at least one electrically insulating contact carrier (42) and at least one conductive, elastic spring contact (43, 44, 45, 46) held thereby which has at least two electrically conductive contacting faces (43.1, 43.2, 44.1, 44.2, 45.1, 45.2, 46.1, 46.2), which are electrically connected to one another which is implemented in such a way that at least one of the contacting faces (43.2, 44.2, 45.2, 46.2) is deflected elastically sprung under pressure on the at least two electrically conductive contacting faces and builds up a defined spring restoring force; and,
an electrical connection being produced between two electrical components by the spring contact block in such a way that a contacting face of the spring contact selected from the at least one conductive, elastic spring contact rests on at least one counter contact face (32.1, 32.2, 32.3, 32.4) of the at least one component and exerts a pressure force generated by the defined spring restoring force of the spring contact on the at least one counter contact face.

2. The medical implant according to claim 1, wherein one of the at least two electrical components is a voltage source (30) and a further of the at least two electrical components is an electrical consumer, and both are electrically connected to one another via the spring contact block (40).

3. The medical implant according to claim 2, wherein both the voltage source (30) and also the electrical consumer (50) have the at least one counter contact face (32.1, 32.2, 32.3, 32.4) compatible with the contacting face (43.2, 44.2, 45.2, 46.2) of the at least one conductive, elastic spring contact (43, 44, 45, 46) and an electrical connection is closed between the electrical consumer (50) and the voltage source (30), both on a side of the voltage source and also on a side of the electrical consumer, with aid of the pressure force caused by the defined spring restoring force of the spring contact between the contacting face of at least one spring contact and the at least one counter contact face.

4. The medical implant according to claim 2, wherein the electrical consumer (50) is connected fixed to the at least one conductive, elastic spring contact (43, 44, 45, 46), and an electrical connection is produced between the electrical consumer and the voltage source (30) with the aid of the pressure force caused by the spring restoring force of the spring contact between the contacting face (43.2, 44.2, 45.2, 46.2) of this spring contact and at least one counter contact face (32.1, 32.2, 32.3, 32.4) of the voltage source.

5. The medical implant according to claim 2, wherein the voltage source (30) is connected fixedly to the at least one conductive, elastic spring contact (43, 44, 45, 46) and an electrical connection is produced between the electrical consumer (50) and the voltage source with the aid of the pressure force caused by the spring restoring force of the spring contact between the contacting face (43.2, 44.2, 45.2, 46.2) of this spring contact and at least one counter contact face (32.1, 32.2, 32.3, 32.4) of the electrical consumer.

6. The medical implant according to claim 1, wherein the liquid-tight housing is assembled from two housing parts (20, 21), one of the housing parts (21) being connected fixedly to the electrical consumer (51).

7. The medical implant according to claim 1, wherein the liquid-tight housing is assembled from two housing parts (20, 21), one of the housing parts (20) being connected fixedly to the voltage source (30).

8. The medical implant according to claim 1, wherein the spring contacts (43, 44, 45, 46) comprise an alloy having good conductivity, preferably a copper-containing alloy.

9. The medical implant according to claim 1, wherein the at least one of the contacting faces (43.2, 44.2, 45.2, 46.2) is coated using a noble metal or an alloy, or gold or a nickel-containing alloy or any combination thereof.

10. The medical implant according to claim 1, wherein the at least one conductive, elastic spring contacts (43, 44, 45, 46) have an essentially flat cross-section.

11. The medical implant according to claim 10, wherein a particular at least one conductive, elastic spring contact (43, 44, 45, 46) has at least one curvature exclusively in one plane, which runs orthogonally to a surface of the at least one conductive, elastic spring contact.

12. The medical implant according to claim 11, wherein the curvature runs in a plane which runs orthogonally to a flat side of the at least one conductive, elastic spring contact (43, 44, 45, 46).

13. The medical implant according to claim 1, wherein a particular at least one conductive, elastic spring contact (43, 44, 45, 46) has at least one curvature having a radius of less than 2 mm.

14. The medical implant according to claim 1, wherein the contacting face (43.2, 44.2, 45.2, 46.2) of the at least one conductive, elastic spring contact is curved convexly in the direction of the counter contact face (32.1, 32.2, 32.3, 32.4).

15. The medical implant according to claim 1, wherein the electrical consumer comprises an integrated circuit.

16. The medical implant according to claim 1, wherein the at least one electrically insulating contact carrier (42) forms a mechanical stop, which prevents relative movements perpendicular or horizontal to an upper surface of the contact carrier.
